(19) United States
(12) Patent Application Publication
(10) Pub. No.: US 2023/0149580 A1
(43) Pub. Date: May 18, 2023

(54) SELF-STERILISING DOOR LEVER HANDLE ASSEMBLY

(71) Applicant: Serhii PASICHNYK, m. Makiivka, Donetska obl (UA)

(72) Inventor: Serhii PASICHNYK, m. Makiivka, Donetska obl (UA)

(21) Appl. No.: 17/916,364

(22) PCT Filed: Mar. 29, 2021
(86) PCT No.: PCT/UA2021/000033

§ 371 Date: Sep. 30, 2022

(30) Foreign Application Priority Data
Mar. 30, 2020 (UA) A 2020 02097

(51) Int. Cl. A61L 2/24; E05B 5/00; E05B 7/00; E05B 47/00; A61L 2/10

(52) U.S. Cl. CPC A61L 2/24; E05B 5/00; E05B 7/00; E05B 47/0012; A61L 2/10; E05B 2047/0017; A61L 2202/122; A61L 2202/11; A61L 2202/14; A61L 2202/121

(57) ABSTRACT

The present invention relates to structures of door locking devices that have a special additional function of self-sterilization, and more particularly, to structures of an electromechanical self-sterilising door handle assembly intended for mounting on latches or mortise locks with a bevelled latch bolt and a door handle spindle. The self-sterilising door lever handle assembly includes a rotation mechanism allowing to rotate handle from a standard operating position into a housing where it is sterilized by ultraviolet radiation and back.

### TECHNICAL FIELD

The present invention relates to structures of door locking devices that have a special additional function, and more particularly, to structures of an electromechanical self-sterilising door handle assembly intended for mounting on latches or mortise locks with a bevelled latch bolt and a door handle spindle.

### BACKGROUND INFORMATION AND PRIOR ART

Door handles greatly accumulate and disseminate pathogens that cause contagious diseases, especially door handles in public access areas. It is necessary therefore, to sanitise door handles regularly. Sanitisation may be achieved in many ways, yet almost all of them have low effect against pathogen transmission due to time intervals between applications. Sanitisation can also be performed by means of sterilisation by ionising or ultraviolet radiation. It is well established that ultraviolet radiation has antiseptic properties. For this reason, germicidal lamps are widely used to sanitise indoor air and surfaces, potable water, as well as to sterilise objects, medical instruments and tools. Said lamps are electrical mercury gas-discharge low-pressure lamps with an envelope of uviol glass or other materials that are able to transmit the required spectrum of ultraviolet radiation. The necessary spectrum of ultraviolet radiation for germicidal lamps minimises ozone generation and harmful effects to human skin and eyes. For this reason, hard ultraviolet is removed from the radiation spectrum, so only the spectral line of soft UV with a wavelength of 253.7 nm remains. This allows minimal ozone generation while using the lamp. Germicidal lamps differ from quartz lamps as a quartz envelope does not restrict hard ultraviolet. The germicidal lamps neutralise a significant part of microorganisms such as bacteria, mould, fungi, yeast, spores, etc. Germicidal lamps are used in different devices, such as bacterial irradiators, bacterial re-circulators, devices for sanitising water, etc. However, radiation from germicidal lamps is dangerous to human eyes and skin. These lamps should only be used with the relevant measures in place to protect human contact with radiation. The sterilisation of door handles using ultraviolet radiation of germicidal lamps can be rather effective, convenient and cost-efficient.

There is a prior general concept of certain functions and structural elements of a self-sterilising door handle assembly disclosed in the US utility patent application US 20140208541 A1 (date of publication: 31 Jun. 2014; IPC (2013): B25G 1/00; B08B 7/04). The general structure of the assembly is shown in FIG. 7, FIG. 8, and FIG. 9 of the description of that particular invention. The aforementioned drawings and description illustrate the concept of creating an electromechanical self-sterilising door handle assembly that uses a germicidal ultraviolet lamp installed inside the assembly's housing. Within the assembly's housing, on a rotation axis, a single solid lever element is installed, two integral parts of which are handle levers with gripping surfaces, said handle levers being set perpendicular to one another. Said lever element of the assembly is so installed that one handle lever is located outside the housing in a horizontal position, while the other handle lever remains in a vertical position inside the housing of the assembly to enable sterilisation by means of radiation from the ultraviolet lamp. Said lever element rotates periodically to replace the handle lever located outside the housing with the lever of the sterilised handle. That is to say, the handle that was outside the assembly's housing, enters the cavity of the housing to be sterilised by ultraviolet radiation. Said lever element periodically rotates 90° in opposite directions. The vertical dimension of the assembly housing is adapted to hold one handle lever above the rotation axis of the lever element and to hold the other handle lever below the rotation axis. Accordingly, the exit aperture of handle levers in the assembly housing has a vertical dimension not less than the total length of two handle levers. Therefore, the area irradiated by ultraviolet rays must also have a vertical dimension not less than the total length of two handle levers, which requires the employ of either a large-sized UV lamp or two similar lamps of lesser size. The description of that patent application also includes an embodiment of the assembly with optional blocking of the door lock, optional use of an electric drive and an electronic control unit, but does not disclose the structure and technical implementations of such features and assembly parts.

The above structure of a self-sterilising door handle assembly has some significant flaws.

In particular, the above described embodiment of the lever element requires a large increase in vertical dimensions of the assembly housing, arranging a large sterilisation area and a significant aperture for the throw of the handle levers. Such a large UV irradiation sterilisation area leads to the necessity of using a comparatively large UV lamp or two similar lamps of lesser size, i.e. it would entail significant cost of manufacturing and eventual use of the device.

Taking into account the gas-discharge nature of germicidal lamps and the fact that they have a significant delay of radiation after activation, with the time of this delay possibly proportional to the sterilisation time of handle levers, the usage of such lamps does not allow for periodic deactivation, i.e. in this case such lamps must operate continuously. It was earlier mentioned that UV lamps produce radiation harmful to human eyesight and skin, and that the consumers must be protected from that radiation.

The embodiment of the door handle assembly described requires a large aperture for the throw of the handle levers. However, no safety measures have been proposed for protection against the ultraviolet radiation that can possibly escape the large aperture. Should, in theory, said problem of protecting the large aperture be decided by structural modifications, this will lead to considerable complication of the device structure, eventual additional increase of its dimensions, decreased reliability of the structure and a significantly higher price of the device.

Moreover, said structure of a self-sterilising door handle assembly does not provide any means of protecting the mechanism for rotating the lever element against external force applied onto the gripping surfaces of the handle levers while they are changing their positions. Such protection can be introduced by way of significant reinforcement of the rotation mechanism to withstand application of significant forces onto its elements, which will obviously lead to significantly increased dimensions of the device and its greatly elevated production cost. The lack of such protection may lead to damage and destruction of the elements of the mechanism, precipitating a device failure. Therefore, the lack of solution to this problem makes it practically impossible to use such a door handle assembly in public places.

The object of the present invention is to create a new structure of a self-sterilising door handle assembly that uses ultraviolet radiation for sterilisation, and that ensures minimal device dimensions, appropriate protection of consumers against ultraviolet radiation, and protection of mechanisms against damage consequent to the application of external forces onto its open movable parts.

The closest prior art of the present invention is the above-described self-sterilising door handle assembly disclosed in the US utility patent application US 20140208541 A1.

The common essential features of the present invention and the closest prior art are as follows: a protective housing with a side aperture; the internal disposition in said housing of a mechanism for advancing and retracting the handle lever through said aperture; the internal disposition in said housing of a sterilisation unit that uses UV radiation; and the internal disposition in said housing of an electronic control unit.

### SUMMARY OF THE INVENTION

The above object is achieved as follows.

The self-sterilising door lever handle assembly, comprising a protective housing with a side aperture, a mechanism, disposed inside said housing, for advancing and retracting a handle lever through said aperture, an ultraviolet sterilisation unit disposed inside said housing, and an electronic control unit also disposed inside said housing, in accordance with the present invention, said self-sterilising door lever handle assembly has a unit for rotating the handle levers as the mechanism for advancing and retracting the handle lever through said aperture, and further includes a lock blocking unit disposed in said housing.

The unit for rotating the handle levers is disposed in the lower part of the housing. The lock blocking unit is disposed on the side of the unit for rotating the handle levers. The sterilisation unit is disposed above the unit for rotating the handle levers, at the level of the handle levers in their vertical position.

Also, the unit for rotating the handle levers comprises a supporting rotatable sleeve, that serves as the basis of a mechanism for rotating the handle frontal lever and a mechanism for rotating the handle back lever. The elements of those mechanisms (said elements rotating around the supporting rotatable sleeve) are symmetrically arranged in parallel planes, said planes being perpendicular to the symmetry axis of the supporting rotatable sleeve.

Below the supporting rotatable sleeve there are two safety devices, through which the parts of the mechanism for rotating the handle frontal lever and the mechanism for rotating the handle back lever (said parts of the mechanisms directly rotating around the supporting rotatable sleeve) are connected to individual electrical rotation devices through gear transmissions. I.e. every mechanism for rotating the handle lever comprises its own separate safety device and its own separate electrical rotation device. Said safety devices are capable of ensuring forced rotation of the mechanism's elements, should any additional loads thereon occur from the mechanism's elements that directly rotate around the supporting rotatable sleeve.

The supporting rotatable sleeve is rotatably mounted and has a co-axial aperture with a cross section adapted for coupling to a spindle of the locking device and for rotating the supporting rotatable sleeve together with said spindle. The supporting rotatable sleeve is connected to a return spring configured to return this sleeve into its initial position after rotation.

The above-described parts of the mechanisms for rotating the handle levers that directly rotate around the supporting rotatable sleeve are manifested in the form of a gear of the handle frontal lever, said gear being rigidly connected to the handle frontal lever, and in the form of a gear of the handle back lever, said gear being rigidly connected to the handle back lever.

The unit for rotating the handle levers has a means of securing the handle levers relative to the supporting rotatable sleeve enabling said sleeve to rotate when the grasping portions of said levers are pressed, said means of securing the handle levers being implemented by ensuring the interaction of special ridges on the surfaces of the supporting rotatable sleeve, handle frontal lever, and handle back lever.

Additionally, the unit for rotating the handle levers is provided with a device for detecting the position of handle levers.

The above-mentioned lock blocking unit is manifested in the form of a mechanical locking device with a latch that blocks the rotation of the supporting rotatable sleeve, the movement of this latch is enabled by a respective electric drive.

The abovementioned sterilisation unit contains a germicidal lamp with ultraviolet radiation, an electrical rotation device, a movable mirror, a fixed mirror, and a movable curtain for closing the larger portion of said side aperture in the housing. The fixed mirror is disposed facing the germicidal lamp and on the opposite side (relative to said lamp) from the area in which the grasping portions of the handle levers are disposed in their vertical position. The movable mirror in its operating position is disposed on the opposite side from the germicidal lamp (relative to the area in which the grasping portions of the handle levers are disposed in their vertical position). The movable mirror and the fixed mirror are disposed to be capable of reflecting and directing the radiation of the germicidal lamp onto the area in which the grasping portions of handle levers are disposed in their vertical position. The movable mirror is configured to rotate around the germicidal lamp in order to prevent radiation from the germicidal lamp leaking through the open side aperture of the housing. The movable curtain is configured to move whereby opening and closing a larger portion of the side aperture in the housing to prevent radiation from the germicidal lamp leaking outside the housing. The above movable mirror and the movable curtain are mechanically coupled to the electrical rotation device of the sterilisation unit and are synchronised so that when the movable curtain moves to open the side aperture in the housing, the movable mirror rotates around the germicidal lamp, preventing radiation from the germicidal lamp leaking through the open side aperture of the housing. And vice versa, when the movable curtain moves to close the side aperture in the housing, the movable mirror rotates and returns to its operating position.

The self-sterilising door lever handle assembly is so configured that, while in the static state, one of the above-mentioned handle levers remains in its operating position to enable securing this lever relative to the supporting rotatable sleeve and to rotate the latter when the grasping portion of this handle lever protruding outside the housing through the side aperture is pressed. Meanwhile, the second handle lever is disposed in the sterilisation position, i.e. inside the housing in the sterilisation unit area, the movable mirror is disposed in the above-mentioned operating position, and the side aperture in the housing is closed by the movable curtain.

The electronic control unit, mentioned above, is embodied in the form of a printed circuit board that comprises a microcontroller, said electronic control unit being connected to the above device for detecting the position of handle levers, electrical rotation devices of the unit for rotating the handle levers, electric drive of the lock blocking unit, and electrical rotation device of the sterilisation unit.

The electronic control unit is configured to detect the position of the handle levers using a device for detecting the position of handle levers.

Also, the electronic control unit is configured to issue rotate commands, any of both directions, to the electrical rotation devices of the unit for rotating the handle levers and the electrical rotation device of the sterilisation unit, as well as block and unblock commands to the electric drive of the lock blocking unit.

Besides, the electronic control unit is configured to issue separate sequential rotate commands to the electrical rotation devices of the unit for rotating the handle levers that enable one handle lever to turn from the operating position to the sterilisation position, followed by the turn of the other handle lever from the sterilisation position to the operating position.

The electronic control unit is configured to issue said rotate commands to the electrical rotation devices of the unit for rotating the handle levers in a programmable time interval and/or after detecting the change in position of the handle lever that is in the operating position as a result of applying external forces onto the latter, with the supporting rotatable sleeve set in rotation.

Besides, the electronic control unit is configured to issue a rotate command to the electrical rotation device of the sterilisation unit in order to open the side aperture in the housing, before the above rotate commands are issued to the electrical rotation devices of the unit for rotating the handle levers.

Also, the electronic control unit is configured to issue an opposite direction rotate command to the electrical rotation device of the sterilisation unit in order to close the side aperture in the housing after completion of previous rotate commands, issued to the electrical rotation devices of the unit for rotating the handle levers.

In addition, the electronic control unit is configured to issue a block command to the electric drive of the lock blocking unit once it is detected that one of the handle levers is in a static position that corresponds to the “blocking” position, and to issue an unblock command once it is detected that one of the above handle levers is in the operating position.

In some embodiments of the invention, the means of securing the handle levers relative to the supporting rotatable sleeve may be implemented by providing the supporting rotatable sleeve with a support pin on its outer surface, and by providing the handle levers with thrust pins. Said thrust pins may be disposed to enable leaning on said support pin and holding the relevant handle levers in a horizontal position, and to enable rotation of the supporting rotatable sleeve when the grasping portions of said levers are pressed with a force exceeding the resistance of the above-mentioned return spring of the supporting rotatable sleeve.

Each safety device of the unit for rotating the handle levers may be of a spring safety coupling type and be assembled on a shaft configured to rotate so that its rotation axis be parallel to the rotation axis of the supporting rotatable sleeve. A drive's gear may be rigidly mounted on each such shaft, said drive's gear being driven relative to the relevant electrical rotation device of this unit. A gear of the safety coupling, that is a master gear relative to the respective above-mentioned gear of the handle lever, is rotatably mounted and abutts with one of its end-faces against the thrust collar of the shaft. A half-coupling may be installed directly against the second end face of this master gear, clamped with a spring. The matching end-faces of the gear of the safety coupling and those of the half-coupling are provided with correspondent matching mortices, with balls disposed between said mortises. The half-coupling may be provided with a feature preventing rotation relative to the shaft's surface.

I.e., the half-coupling may be configured to move along the shaft if there is a force that exceeds the force of the spring clamping of said half-coupling to said master gear, and to return into its initial state when said force ceases. The gear of the safety coupling is configured to rotate in conjunction with the shaft, on which it is mounted, and to transmit the torque of the gear of the relevant handle lever if there is no external force that exceeds the force of the spring clamping of said half-coupling.

The above-mentioned device for detecting the position of handle levers of the unit for rotating the handle levers may be configured on the basis of a magnetic resistant sensor and be disposed inside the supporting rotatable sleeve. Said device for detecting the position has two sensing elements with one magnet in a non-metal sleeve opposing each of the sensing elements. Each non-metal sleeve is rigidly connected to one of the above-mentioned handle levers, enabling simultaneous rotation around the symmetry axis of the supporting rotatable sleeve together with a relevant handle lever. Said connection of each non-metal sleeve may be carried out via a bracket of the relevant handle lever, drawn through the slot opening in a wall of the supporting rotatable sleeve. Each said bracket extends outside the supporting rotatable sleeve through a slot opening and connects to a relevant handle lever. Said slot openings of the supporting rotatable sleeve are configured to enable a 90° rotation of said brackets.

The electrical rotation devices of the unit for rotating the handle levers may be embodied as servo drives with a latch for securing the rotor's position when an engine does not operate. The rotor of each said servo drive is rigidly connected to a gear that forms a gear transmission with the relevant driven gear of the above-mentioned safety device of the unit for rotating the handle levers.

Also, in a specific embodiment, the electric drive of the lock blocking unit may be configured as a servo drive, with a rotor rigidly connected to an adapter sleeve in which a vertical rotating shaft is rotatably disposed, with a latch rigidly fastened to said shaft. A spring may be installed on said shaft, with one end of said spring fastened on the above adapter sleeve and another end fastened on said shaft. In other words, said rotating shaft may be adapted for rotation due to its connection to a rotor of said servo drive through a spring.

The lock blocking unit may be disposed to enable, while turning into the blocking position, retraction of said latch into a special mortise on the external surface of the above-mentioned supporting rotatable sleeve.

In specific embodiments of the present invention, the sterilisation unit may contain a bracket with a socket, in which the above-mentioned germicidal lamp is installed. Above said germicidal lamp, a fixed bracket may be disposed, and above the latter there is disposed an electrical rotation device configured as a servo drive, the rotor of said servo drive being connected to the master gear of the drive, said master gear being located in the horizontal plane below the housing of said servo drive and configured in the form of a disk mortise cam. Also, a rotating sleeve is rotatably disposed in the aperture of the fixed bracket, and rigidly connected to a movable bracket, with the above-mentioned movable mirror fastened on the lower portion of said movable bracket. Inside the rotating sleeve, offset from its centre, there is disposed a vertical cylindrical pin, with the lower portion thereof coupled to said movable bracket, and with the upper portion thereof disposed in a mortise of said drive's gear. The above-mentioned movable curtain of the sterilisation unit may be embodied as a plate installed in the rails disposed on the inner surface of the sidewall of the housing. I.e., the movable curtain may be configured to move horizontally along the sidewall of the housing. The surface of the movable curtain, facing the housing's cavity, is fitted with two horizontal cogged racks. A vertical rotating shaft is rotatably mounted, beside the movable curtain, on the sidewall of the housing. On the upper portion of said rotating shaft is a rigidly fixed driven gear that forms a gear transmission with the above-mentioned drive's gear and is a driven one. Two gears forming a racked gear transmission with the cogged racks of the movable curtain are rigidly mounted on said rotating shaft.

The sterilisation unit may be so configured that the drive's gear, embodied as a disk mortise cam, when rotating clockwise, moves the above cylindrical pin, which slides in a mortise of said gear. At the same time, the movable bracket rotates with the movable mirror. As the movable mirror rotates into the position of closing the germicidal lamp, the cylindrical pin travels a portion of the mortise of the drive's gear and stops. Further rotation of the drive's gear causes a no-load run of said cam mechanism. That is why the movable mirror does not move until a completion of the full rotation by the drive's gear. Concurrent with the start of rotation of the drive's gear, the above-mentioned driven gear rotates together with the rotating shaft that moves the movable curtain through the above-mentioned cogged racks, so that the side aperture in the housing fully opens in one full rotation of the drive's gear. When the drive's gear rotates in the opposite direction, the movable mirror returns in a reverse order into its initial operating position, and the side aperture in the housing is closed by the movable curtain.

In another specific embodiment, the above-mentioned electronic control unit may be configured to issue a block command to the electric drive of the lock blocking unit once it is detected that one of the handle levers is in a static position at an angle to the horizontal line greater than 0° and lesser than 90°, and to issue an unblocking command once it is detected that one of the above handle levers is in a horizontal position.

Given that the present invention can be modified and have alternative embodiments, it is understood that the bellow detailed description is exemplary of its essential features and implementation. It is further understood that the bellow detailed description is not to be construed as limiting the present invention to certain embodiments detailed below. Instead it includes all modifications, equivalents and alternatives, which do not depart from the spirit and scope of patent protection of the structure of the self-sterilising door lever handle assembly as described and claimed bellow.

### BRIEF DESCRIPTION OF THE DRAWINGS

The key point of the present invention is illustrated in the following drawings:

FIG. 1—Self-sterilising door lever handle assembly (axonometric view 1)

FIG. 2—Self-sterilising door handle assembly (axonometric view 2)

FIG. 3—Unit for rotating the handle levers of the self-sterilising door lever handle assembly (axonometric view 1)

FIG. 4—Unit for rotating the handle levers of the self-sterilising door lever handle assembly (axonometric view 2)

FIG. 5—Unit for rotating the handle levers of the self-sterilising door lever handle assembly (frontal projection)

FIG. 6—Unit for rotating the handle levers of the self-sterilising door lever handle assembly (lateral projection)

FIG. 7—Unit for rotating the handle levers of the self-sterilising door lever handle assembly (back projection)

FIG. 8—Unit for rotating the handle levers of the self-sterilising door lever handle assembly (view A)

FIG. 9—Safety device of the rotation mechanism of the handle back lever

FIG. 10—Safety device of the rotation mechanism of the handle frontal lever

FIG. 11—Unit for rotating the handle levers of the self-sterilising door lever handle assembly (axonometric view 3)

FIG. 12—Unit for rotating the handle levers of the self-sterilising door lever handle assembly (axonometric view 4)

FIG. 13—Lock blocking unit

FIG. 14—Lock blocking unit (View B-B)

FIG. 15—Lock blocking unit (View C-C)

FIG. 16—Lock blocking unit (axonometric view)

FIG. 17—Sterilisation unit

FIG. 18—Sterilisation unit (axonometric view 1)

FIG. 19—Sterilisation unit (axonometric view 2)

FIG. 20—Sterilisation unit (View D-D)

FIG. 21—Sterilisation unit (View E-E)

These drawings do not in any way limit implementation of the claimed structure of the self-sterilising door handle assembly, nor any other alternative embodiments, within the scope of the invention disclosed and claimed herein. The present

drawings illustrate the key point of implementing the invention using an indicative physical object that has features claimed bellow.

### DETAILED DESCRIPTION OF THE EMBODIMENTS

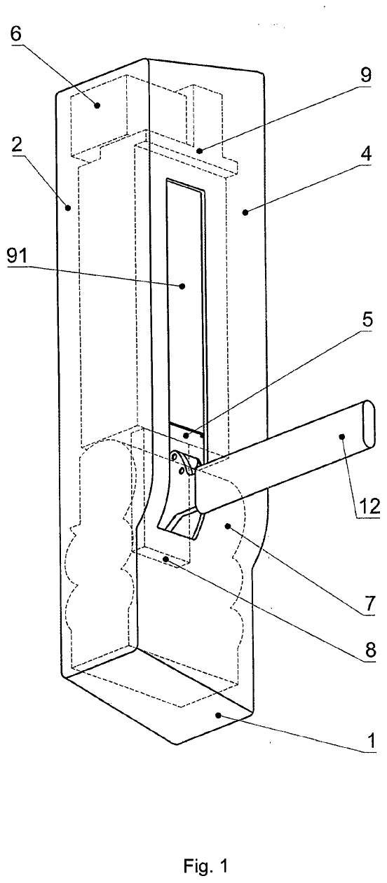

In a specific embodiment, the self-sterilising door lever handle assembly (FIG. 1) comprises a housing 1, said housing 1 comprises the frontal wall 2 and the back wall 3 both of which are similarly shaped, as well as side walls, one of which, namely the wall 4, has a rectangular aperture 5.

Inside the housing 1, disposed are the electronic control unit 6, the unit 7 for rotating the handle levers, the lock blocking unit 8 and the sterilisation unit 9 for handle levers.

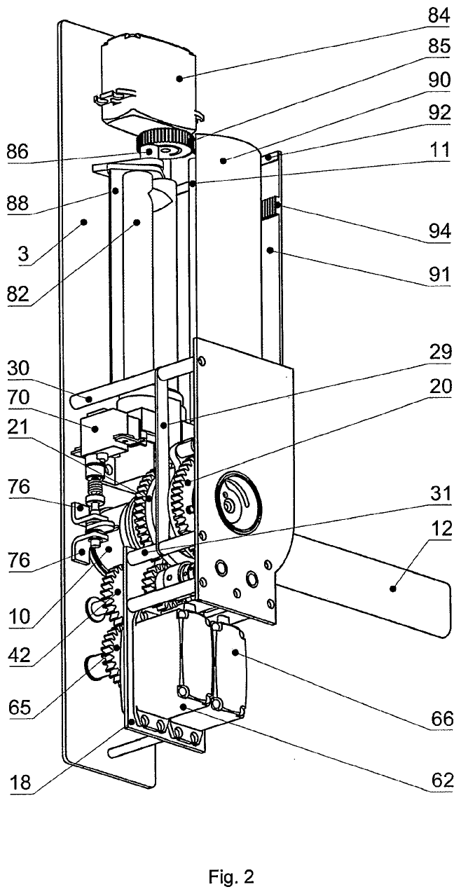

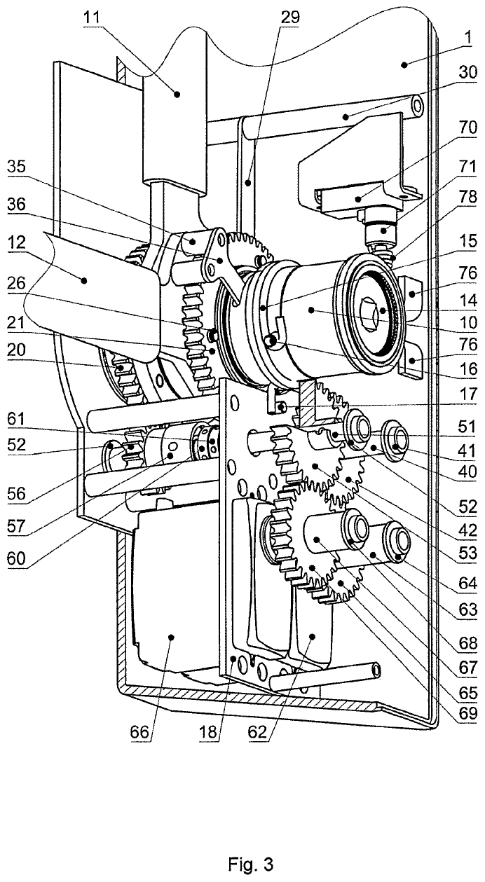

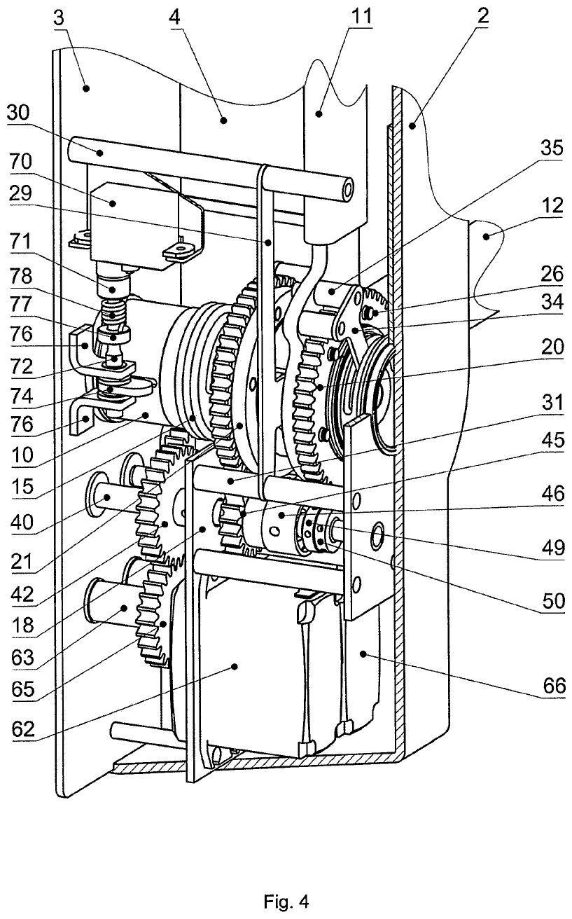

The unit 7 for rotating the handle levers (FIG. 1, FIG. 2, FIG. 3, FIG. 4) is disposed in the lower portion of the housing 1. The lock blocking unit 8 is disposed adjacent to the unit 7 for rotating the handle levers and coupled therewith. The sterilisation unit 9 for handle levers is disposed above the rotatable portion of the unit 7 for rotating the handle levers, at the level of the handle levers in their vertical position. In a specific embodiment, the electronic control unit 6, may be disposed in the upper portion of the housing 1.

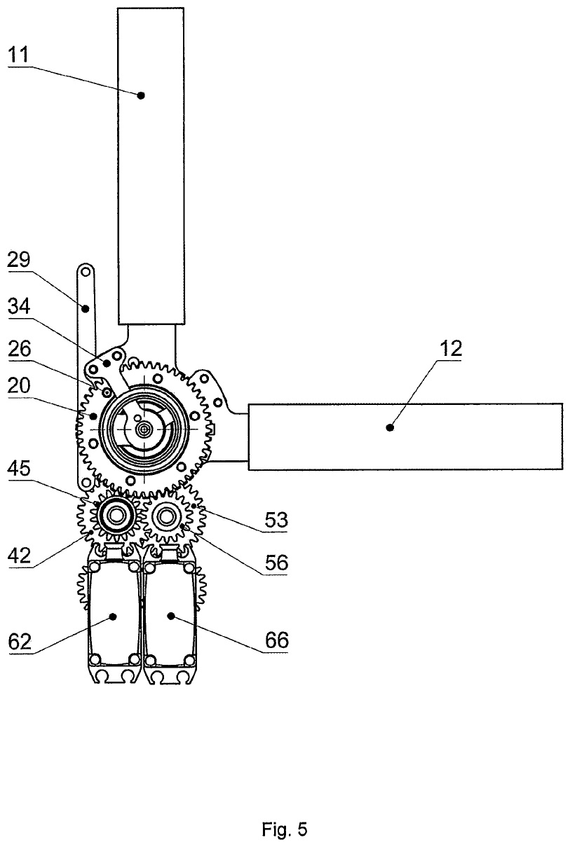

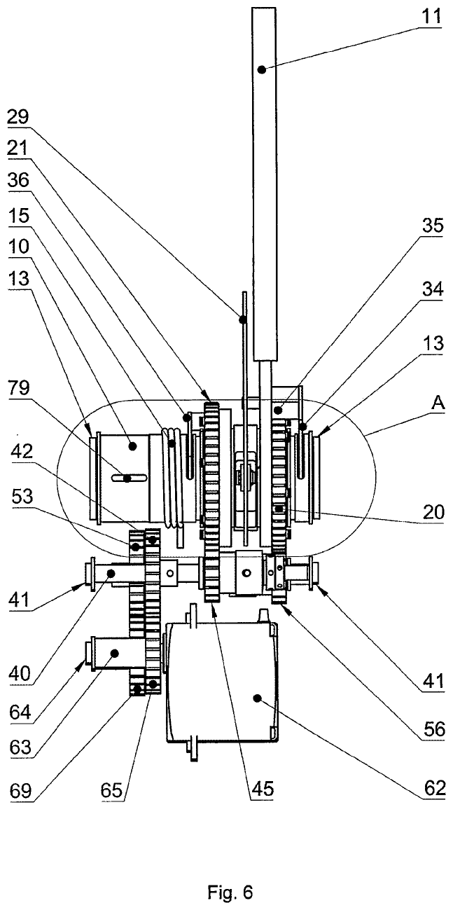

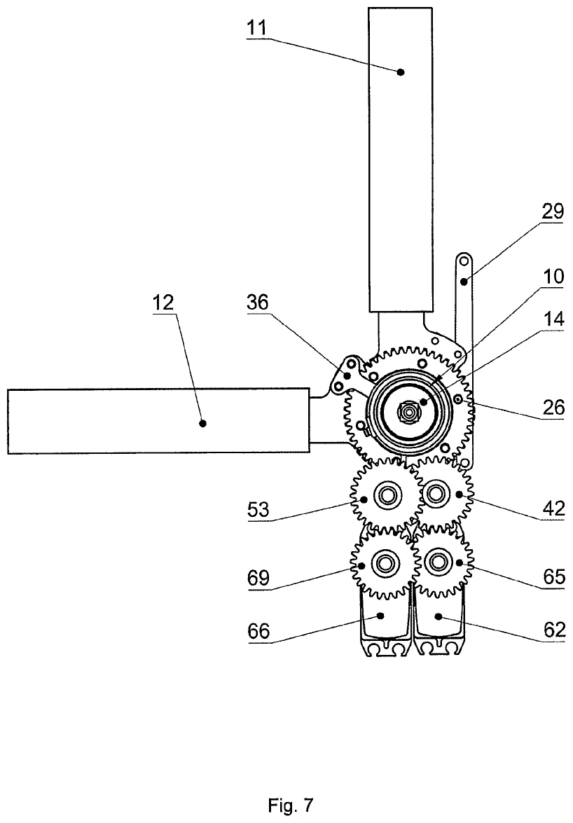

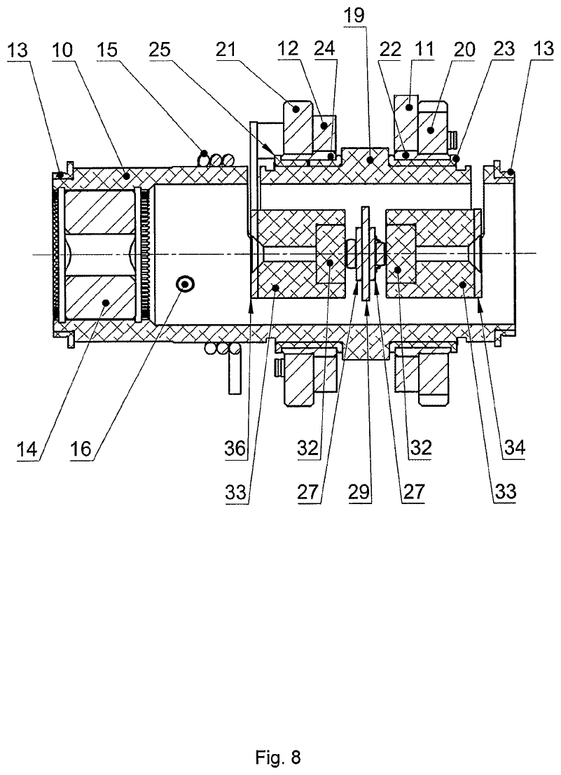

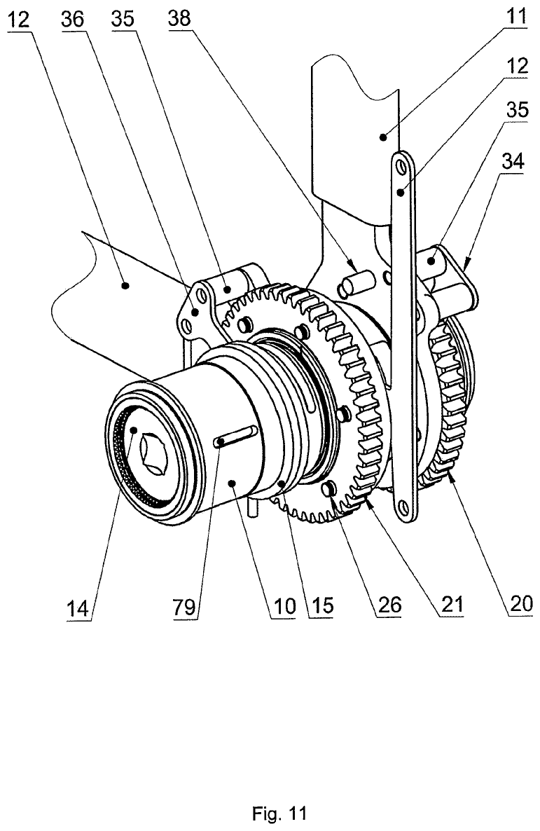

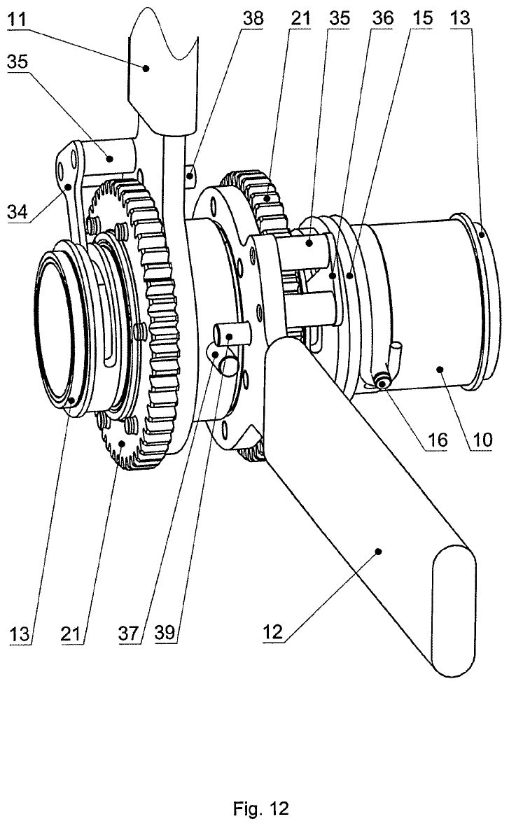

The unit 7 for rotating the handle levers (FIG. 1, FIG. 2, FIG. 3, FIG. 4) comprises the supporting rotatable sleeve 10 (FIG. 5, FIG. 6, FIG. 7, FIG. 8, FIG. 11, FIG. 12), that serves as the basis of the mechanism for rotating the handle 11 frontal lever and the mechanism for rotating the handle 12 back lever. The elements of those mechanisms that directly rotate around the supporting rotatable sleeve 10 are symmetrically arranged in parallel planes, said planes being perpendicular to the symmetry axis of the supporting rotatable sleeve 10. Below the supporting rotatable sleeve 10 there are two safety devices, through which said parts of the mechanism for rotating the handle 11 frontal lever and the mechanism for rotating the handle 12 back lever that both directly rotate around the supporting rotatable sleeve 10 are connected to individual electrical rotation devices through gear transmissions. I.e. every mechanism for rotating the handle lever comprises its own separate safety device and its own separate electrical rotation device. Said safety devices are devised to prevent the movable parts of the mechanisms from overloading that may occur during operation because of external physical influences that may cause damage or destruction to parts of the mechanism. The safety devices mentioned are configured to enable forced rotation of the mechanism's elements, should any certain loads thereon occur from the mechanism's elements that directly rotate around the supporting rotatable sleeve 10.

The above-mentioned supporting rotatable sleeve 10 is rotatably disposed in the housing 1 by way of inserting its ends into sliding sleeves 13, one of said sliding sleeves 13 being disposed in the aperture of the back wall 3, and another one being fastened to an inner surface of the frontal wall 3 of said housing 1.

A co-axial coupling sleeve 14 is rigidly fastened inside the supporting rotatable sleeve 10, on the side of the back wall 3 (FIG. 8, FIG. 11), in order to join to a spindle (not shown in the drawings) of the locking device (not shown in the drawings). This coupling sleeve 14 has a through aperture with a cross-section adapted for coupling to said spindle and for rotating the supporting rotatable sleeve 10 together with said spindle. In this particular embodiment, the sleeve 14 has a square aperture adapted for coupling with a conventional spindle of the locking device that has an 8 mm square cross section. The rigid fastening of the coupling sleeve 14 inside the supporting rotatable sleeve 10, mentioned above, may be implemented, in particular, by press-fitting a screw connection or a keyed connection.

A spiral return spring 15 is installed on the outer surface of the supporting rotatable sleeve 10 (FIG. 3, FIG. 8, FIG. 11) to ensure that the supporting rotatable sleeve 10 returns to its initial position after rotation. One end of the return spring 15 is connected to the supporting rotatable sleeve 10, in this embodiment—by means of hitching to the pin 16 located on the outer surface of this sleeve. Meanwhile, the second end of said return spring 15 is connected to the housing 1, in this embodiment—by means of hitching to the pin 17 of the supporting plate 18. Thus, due to the return spring 15, while there is no external force applied, the position of the supporting rotatable sleeve 10 is secured, together with all the elements of mechanisms that have a fixed connection to said sleeve 10.

As stated above, on the supporting rotatable sleeve 10 disposed are portions of mechanisms for rotating the handle 11 frontal lever and for rotating the handle 12 back lever installed (FIG. 8, FIG. 11, FIG. 12), that directly rotate around said sleeve and are arranged in parallel planes symmetrically to one another. In this particular embodiment, said symmetry is achieved by installing said parts of the mechanisms on both sides of the distance spacer, said distance spacer being embodied in the form of a spacing collar 19 of the outer surface of the supporting rotatable sleeve 10. The gear 20 of the handle 11 frontal lever, is rotatably mounted from one side of the collar 19, and the gear 21 of the handle 12 back lever is rotatably mounted from the other side. The gear 20 of the handle 11 frontal lever has a co-axial supporting sleeve 22, welded to it, by means of which it is mounted on the supporting rotatable sleeve 10 via the sliding sleeve 23 of the handle 11 frontal lever. Similarly, the gear 21 of the handle 12 back lever has a co-axial supporting sleeve 24, welded to it, by means of which it is mounted on the supporting rotatable sleeve 10 via the sliding sleeve 25 of the handle 12 back lever.

The gear 20 of the handle 11 frontal lever is rigidly connected to the handle 11 frontal lever, and the gear 21 of the handle 12 back lever is rigidly connected to the handle 12 back lever. In this embodiment, each lever of the handle has a grasping portion that is connected to a ring-like portion for mounting on the supporting rotatable sleeve 10. The grasping portion of each lever of the handle may be manifested in the form of a grip of any form dependent on ergonomic and aesthetic needs. The handle 11 frontal lever is connected to the gear 20 of this lever by means of a tight installation of said ring-like portion of said lever on the above-mentioned supporting sleeve 22 of said gear, closely to the lateral face of said gear, and fastened by pins 26. Similarly, the handle 12 back lever is connected to the gear 21 of said lever by means of a tight installation of the ringlike portion of said lever on the above-mentioned supporting sleeve 24 of said gear, closely to the lateral face of this gear, and fastened by pins 26.

A device for detecting the position of the handle 11 frontal lever (FIG. 8) and handle 12 back lever is disposed inside the supporting rotatable sleeve 10. In this particular embodiment, said device for detecting the position of handle levers is configured on the basis of the magnetic resistant sensor. Said device is disposed co-axially with the supporting rotatable sleeve 10 and has two sensing elements of the sensor 27, that are symmetrically arranged on both sides of the bracket's 29 plate. Said bracket's 29 plate is disposed perpendicular to the symmetry axis of the supporting rotatable sleeve 10 and is inserted into the cavity inside said sleeve through a slot opening in its wall (FIG. 4, FIG. 8), while outside said sleeve the bracket 29 is connected to the housing 1 through the coupling sleeve 30 and connected to the above-mentioned supporting plate 18 through the coupling sleeve 31. Said slot opening, through which the bracket's 29 plate is installed, is configured to enable a rotation of the sleeve 10 at an angle sufficient for normal rotation of the spindle of the locking device. Opposing each of said sensing elements of the sensor 27, there is a magnet 32 press-fitted into the non-metal supporting sleeve 33. Each supporting sleeve 33 is rigidly connected to one of the above-mentioned handle levers. That is to say, each supporting sleeve, together with the magnet 32 installed therein, is adapted to enable simultaneous rotation around the symmetry axis of the supporting rotatable sleeve 10 together with a respective handle lever. In this particular embodiment, said connection is implemented via a bracket of a relevant handle lever, drawn through the slot opening in the wall of the supporting rotatable sleeve 10. Further, one supporting sleeve 33 with its end face opposing the magnet's 32 disposition is connected by a screw to the plate-like bracket 34 that goes outwards from the supporting rotatable sleeve 10 through a slot opening, and, by means of supporting sleeves 35, is connected to the handle 11 frontal lever. Similarly, the other supporting sleeve 33 with its end face opposing the magnet's 32 disposition is connected by a screw to the plate-like bracket 36 that goes outwards from the supporting rotatable sleeve 10 through a slot opening, and, by means of supporting sleeves 35, is connected to the handle 12 back lever. Importantly, said slot openings of the supporting rotatable sleeve 10 are configured to allow a 90° rotation of the bracket 34 and the bracket 36.

The unit 7 for rotating the handle levers has a means of securing the handle levers relative to the supporting rotatable sleeve 10 enabling said sleeve 10 to rotate when the grasping portions of these levers are pressed. Said means of securing the handle levers is implemented by ensuring the interaction of special ridges on the surfaces of the supporting rotatable sleeve 10, the handle 11 frontal lever and the handle's 12 back lever. The supporting rotatable sleeve 10 (FIG. 11, FIG. 12) is configured with a support pin 37 on its outer surface. The handle 11 frontal lever is configured with a thrust pin 38, and the handle 12 back lever is configured with a thrust pin 39. The thrust pin 38 and the thrust pin 39 are disposed to enable leaning on the support pin 37 and holding the relevant handle levers in a horizontal position, and to enable rotation of the rotatable sleeve 10, when the grasping portions of these levers are pressed with a force exceeding the resistance of the above-mentioned return spring 15.

Below the supporting rotatable sleeve 10 there are two safety devices, each of which, in this embodiment, is configured on the basis of the spring safety coupling which limits the torque that may be transmitted from the above handle levers, thus protecting a relevant electrical rotation device from overload and destruction.

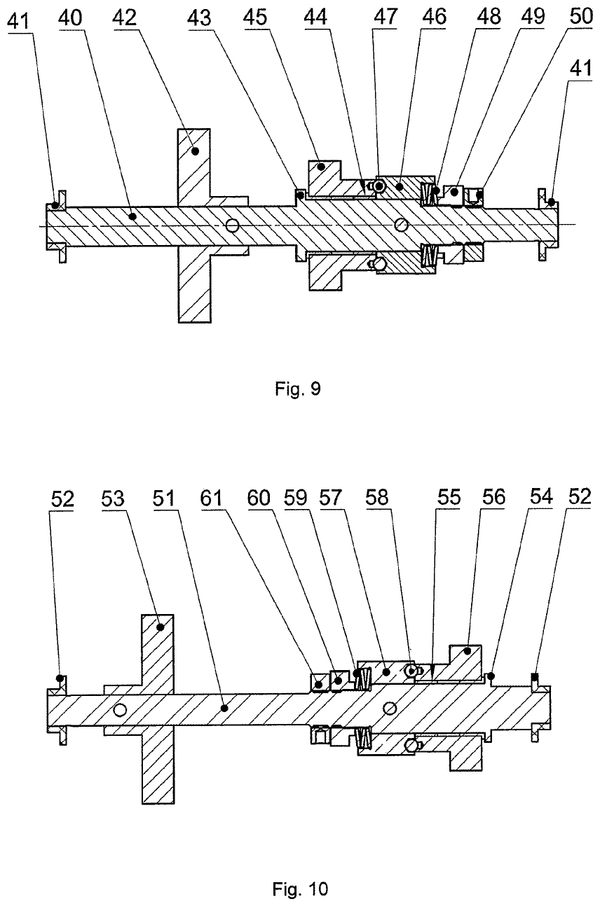

The safety device of the mechanism for rotating the handle 12 back lever (FIG. 9) is assembled on the shaft 40, said shaft being disposed in the housing 1 and adapted to rotate by way of inserting its ends into sliding sleeves 41, one of which is installed in the aperture of the back wall 3, and another is fastened to the inner surface of the frontal wall 3 of the housing 1. The shaft 40 of the safety device is so disposed that its rotation axis is parallel to the rotation axis of the supporting rotatable sleeve 10. The driven gear 42 of the drive is rigidly mounted onto the shaft 40, on the side of the back wall 3 of the housing 1. The outer surface of the shaft 40 comprises a thrust collar 43, with a gear 45 of the safety coupling installed closely to it, on the side opposite to the gear 42, on the sliding ring 44. The gear 45 of the safety coupling forms a cylindrical gear transmission with the gear 21 of the handle 12 back lever. A half-coupling 46 is mounted on the shaft 40, directly against the gear 45, on the side opposite to the collar 43. The matching end-faces of the half-coupling 46 and the gear 45 are provided with correspondent matching mortises, with balls 47 disposed between said mortises. A spring 48 is mounted on the end-face of the half-coupling 46, said end-face being opposite to said mortises, said spring 48 being secured with an adjusting nut 49, said adjusting nut 49 being secured with a jam nut 50. Said spring 48 is mounted as a safeguard against rotation of the half-coupling 46 around the shaft 40. Consequently, the half coupling 46 is pressed to the gear 45 of the safety coupling with the spring 48 and can move along the shaft 40 if a force exceeding the resistance of said spring 48 is applied, and can return into its initial position after said force ceases. The above-mentioned gear 45 of the safety coupling is adapted to rotate around the shaft 40, if torque occurs from the gear 21 of the handle 12 back lever and the respective force is transmitted to the half-coupling 46 through the balls 47, exceeding the resistance of the spring 48. So, the gear 45 of the safety coupling is adapted to rotate in conjunction with the shaft 40 and transmit the torque of the gear 21 of the handle 12 back lever, while there is no external force applied exceeding the resistance of the spring 48.

The safety device of the mechanism for rotating the handle 11 frontal lever (FIG. 10) is configured similarly to the safety device of the mechanism for rotating the handle 12 back lever, but with a different arrangement of some of the elements, caused by the different disposition of the gear 20 of the handle 11 frontal lever. Further, the safety device of the mechanism for rotating the handle 11 frontal lever is assembled on the shaft 51, said shaft being disposed in the housing 1 and adapted to rotate by way of inserting its ends into sliding sleeves 52, one of which is installed in the aperture of the back wall 3, and another is fastened to the inner surface of the frontal wall 2 of the housing 1. The shaft 51 of the safety device is so disposed that its rotation axis is parallel to the rotation axis of the supporting rotatable sleeve 10. The driven gear 53 of the drive is rigidly mounted onto the shaft 51, on the side of the back wall 3 of the housing 1. The outer surface of the shaft 51 comprises a thrust collar 54, with a gear 56 of the safety coupling installed closely to it, on the side of the gear 53, on the sliding ring 55. The gear 56 of the safety coupling forms a cylindrical gear transmission with the gear 20 of the handle 11 frontal lever. The half-coupling 57 is mounted on the shaft 51, directly against the gear 56, on the side opposite to the collar 54. The matching end-faces of the half-coupling 57 and the gear 56 are provided with correspondent matching mortises, with balls 58 disposed between said mortises. A spring 59 is mounted on the end-face of the half-coupling 57, said end-face being opposite to said mortises, said spring 59 being secured with an adjusting nut 60, said adjusting nut 60 being secured with a jam nut 61. Said spring 59 is mounted as a safeguard against rotation of the half-coupling 57 around the shaft 51. Thus, the half-coupling 57 is pressed to the gear 56 of the safety coupling with a spring 59 and can move along the shaft 51, if a force exceeding the resistance of said spring 59 is applied, and can return into its initial position after said force ceases. The above-mentioned gear 56 of the safety coupling is adapted to rotate around the shaft 51, if torque occurs from the gear 20 of the handle 11 frontal lever and the respective force is transmitted to the half-coupling 57 through the balls 58, exceeding the resistance of the spring 59. So, the gear 56 of the safety coupling is adapted to rotate in conjunction with the shaft 51 and transmit the torque of the gear 20 of the handle 11 frontal lever, with no external force exceeding the resistance of the spring 59 being applied.

Below said safety devices of mechanisms for rotating the handle 11 frontal lever and the handle 12 back lever are disposed the relevant electrical rotation devices, each of which, in this embodiment, is configured as a servo drive with a magnetic latch that secures the position of a rotor when the motors are inactive. The

housings of the above servo drives are fastened to the above-mentioned supporting plate 18 that is connected to the housing 1.

The servo drive 62 (FIG. 2, FIG. 3, FIG. 4, FIG. 5, FIG. 6, FIG. 7) of the mechanism for rotating the handle 12 back lever comprises a shaft 63, the end of which is installed in the sliding sleeve 64, said sliding sleeve 64 being disposed in the aperture in the back wall 3 of the housing 1. The servo drive 62 is disposed so that the rotation axis of its shaft 63 be parallel to the rotation axis of the shaft 40 of the safety device of the mechanism for rotating the handle 12 back lever. The master gear 65 of the drive is rigidly installed on the shaft 63, on the side of the back wall 3 of the housing 1, said master gear 65 forming a cylindrical gear transmission with a driven gear 42 of the shaft 40 drive of the safety device of the mechanism for rotating the handle 12 back lever.

Likewise, the servo drive 66 (FIG. 2, FIG. 3, FIG. 4, FIG. 5, FIG. 6, FIG. 7) of the mechanism for rotating the handle 11 frontal lever comprises a shaft 67, the end of which is installed in the sliding sleeve 68, said sliding sleeve 68 being disposed in the aperture in the back wall 3 of the housing 1. The servo drive 66 is disposed so that the rotation axis of its shaft 67 be parallel to the rotation axis of the shaft 51 of the safety device of the mechanism for rotating the handle 11 frontal lever. The master gear 69 of the drive is rigidly installed on the shaft 67, on the side of the back wall 3 of the housing 1, said master gear 69 forming a cylindrical gear transmission with a driven gear 53 of the shaft 51 drive of the safety device of the mechanism for rotating the handle 11 frontal lever.

Aiming to make the overall dimensions smaller and to make a more compact arrangement, all of the above-mentioned pairs of the master and driven gears are disposed with an offset in parallel planes.

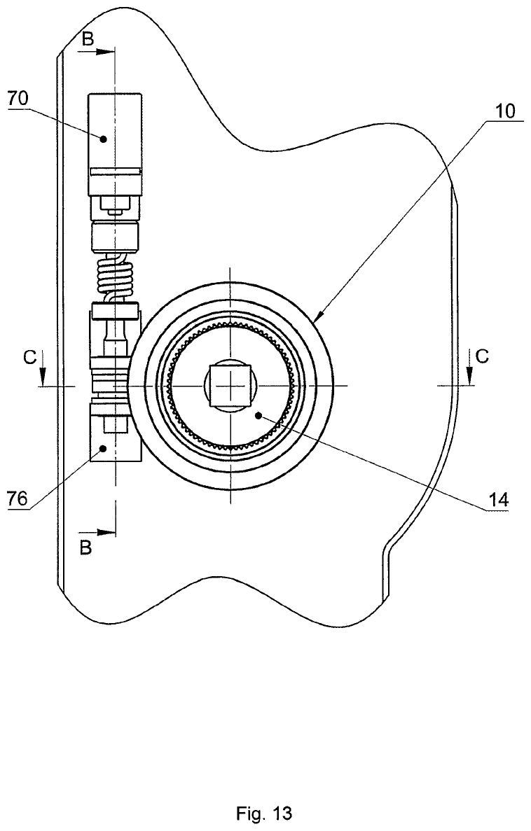

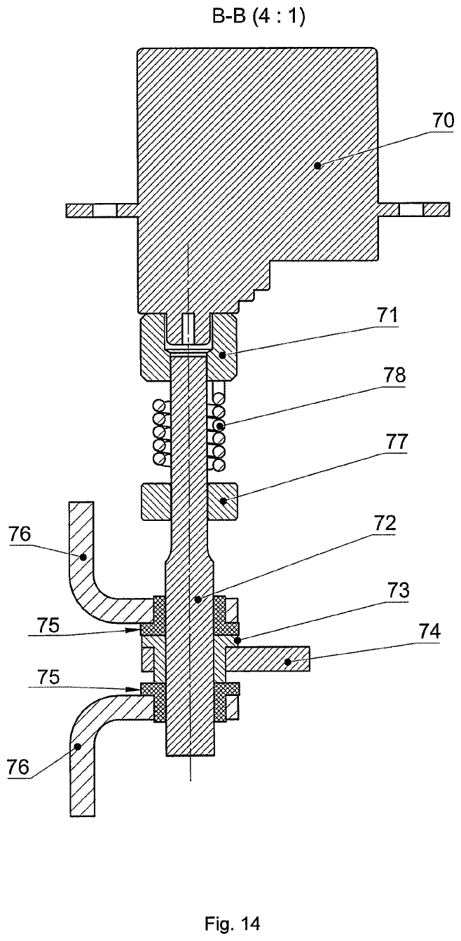

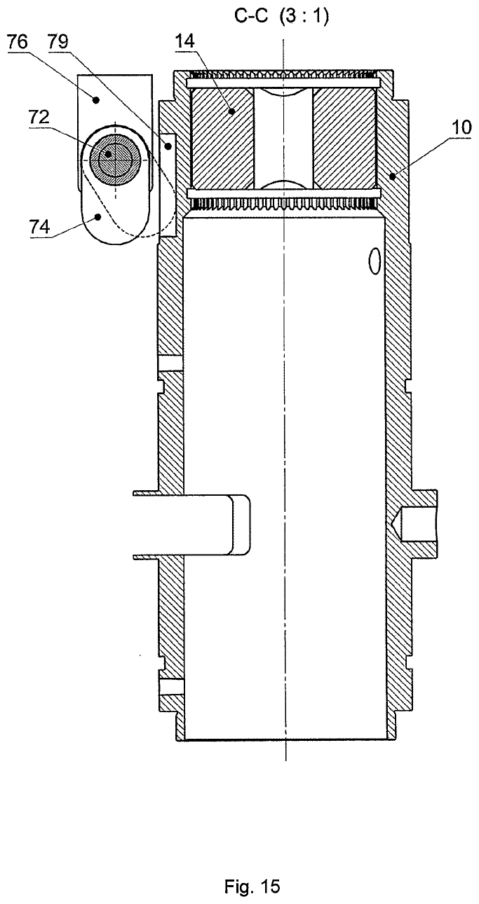

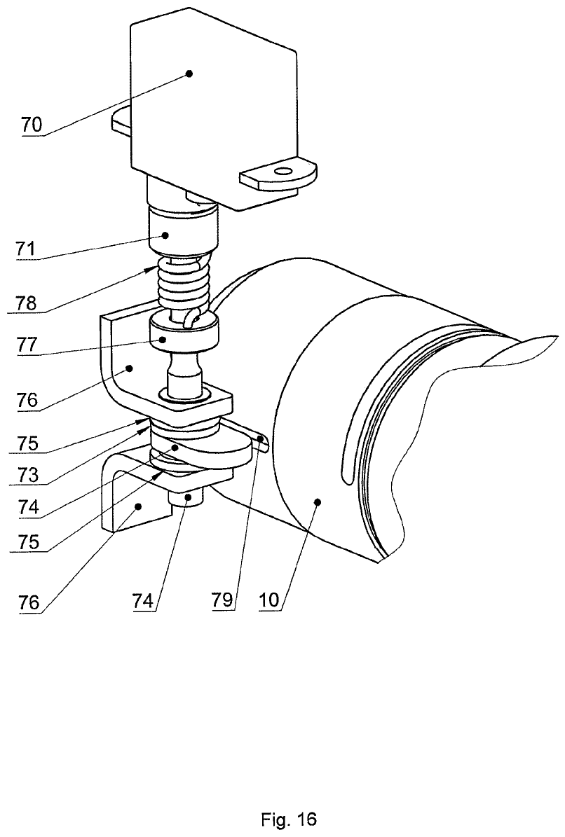

The above-mentioned lock blocking unit 8 (FIG. 13, FIG. 14, FIG. 15, FIG. 16) is configured as a mechanical locking device with a latch that blocks the rotation of the supporting rotatable sleeve 10, while the movement of said latch is enabled by a relevant electric drive. Said locking device is disposed beside the unit 7 for rotating the handle levers and is mounded on the inner surface of the back wall 3 of the housing 1, beside the supporting rotatable sleeve 10.

The lock blocking unit 8 comprises the blocking servo drive 70 fastened to the back wall 3 of the housing 1, said blocking servo drive 70 serving as an electric drive for the lock blocking unit 8. An adapter sleeve 71 of the servo drive is

disposed in the lower part of the servo drive's 70 housing, said adapter sleeve 71 being rigidly connected to the servo drive's rotor. A vertically positioned blocking rotatable shaft 72 is rotatably installed in the sleeve 71 of the servo drive. The lower part of the blocking rotatable shaft 72 is rigidly connected to a sleeve 73 of the latch, to which is welded the blocking latch 74 that is configured as a plate having a rounded end located on the horizontal plane. Further, the lower part of the blocking rotatable shaft 72 is rotatably inserted in two sliding sleeves 75, said sliding sleeves 75 being disposed directly against the end-faces of the sleeve 73 of the latch and inserted into the brackets 76 of the blocking unit, said brackets 76 being fastened to the back wall 3 of the housing 1. A spring's sleeve 77 is disposed in the central part of the blocking rotatable shaft 72, said sleeve 77 being rigidly connected to rotatable shaft 72. The blocking rotatable shaft 72 has a spiral spring 78 installed thereon between the spring's sleeve 77 and the servo drive's adapter sleeve 71, the ends of said spiral spring 78 being secured in the apertures of said sleeves. That is, the blocking rotatable shaft 72 is rotatably mounted due to connection to the rotor of the blocking servo drive 70 through (i) an adapter sleeve 71 of the servo drive, (ii) a spring 78 and (iii) a spring's sleeve 77. Said lock blocking unit 8 is disposed to enable the above-mentioned blocking latch 74, when turned to the blocking position, entering a special horizontal mortise 79, said horizontal mortise 79 being manifested on the outer surface of the supporting rotatable sleeve 10.

As stated above, the return spring 15 secures the position of the supporting rotatable sleeve 10, while there are no external forces applied. Once external forces are applied to open the door, the supporting rotatable sleeve 10 may rotate. If the lock blocking is underway at that time, i.e. the blocking latch 74 is being rotated, said latch will not enter the mortise 79 but will hit the outer surface of the supporting rotatable sleeve 10. Therefore the lock blocking unit 8 is adapted to prevent overloading and destructing elements of the mechanism of the lock blocking unit 8, and to enable automatic travel of the blocking latch 74 into the mortise 79 of the supporting rotatable sleeve 10. Specifically for that purpose, the above blocking shaft 72 is connected to the servo drive 70 through the spring 78. The automatic travel of the blocking latch 74 into the mortice 79 of the supporting rotatable sleeve 10 is enabled by the spring 78, in cases when the supporting rotatable sleeve 10 automatically returns to its normal position under the influence of the return spring 15, once the external forces acting on the supporting rotatable sleeve 10 cease.

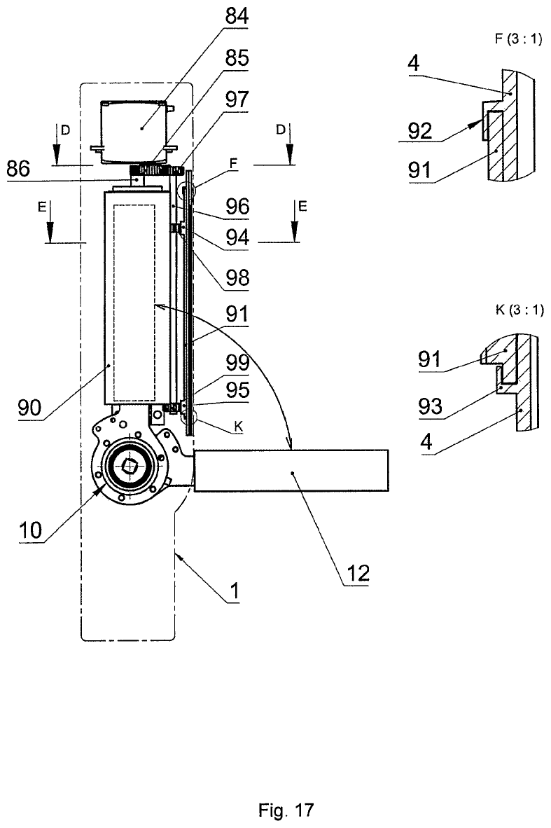

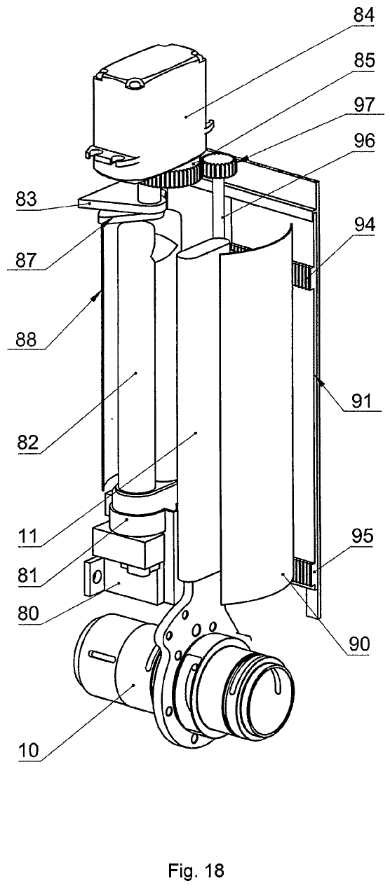

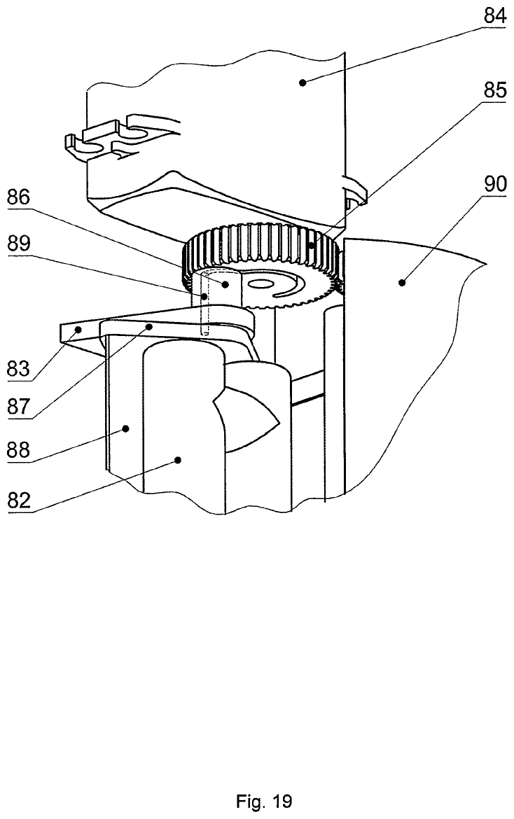

The sterilisation unit 9 for handle levers (FIG. 17, FIG. 18, FIG. 19) comprises a lamp bracket 80, fixed on the back wall 3 of the housing 1. The lamp bracket 80 houses a socket 81, into which a germicidal lamp 82 is inserted.

In this particular embodiment of the present invention, a standard germicidal lamp PHILIPS TUV PL-S 7 W/2P 1CT with a cap base G23 is used in the unit 9 for sterilising the handle levers, for the germicidal lamp 82. Germicidal lamps are gas-discharge lamps that have a significant delay of radiation after activation with the time of such delay possibly being proportional to the sterilisation time of handle levers, therefore such lamps should only be used in a continuous mode, i.e. without periodic deactivations. In order to enhance the effect of the lamp 82, the grasping portions of the above-mentioned handle 11 frontal lever and handle 12 back lever may be provided with a surface or coating that contains catalytic or photocatalytic agents, particularly titanium dioxide.

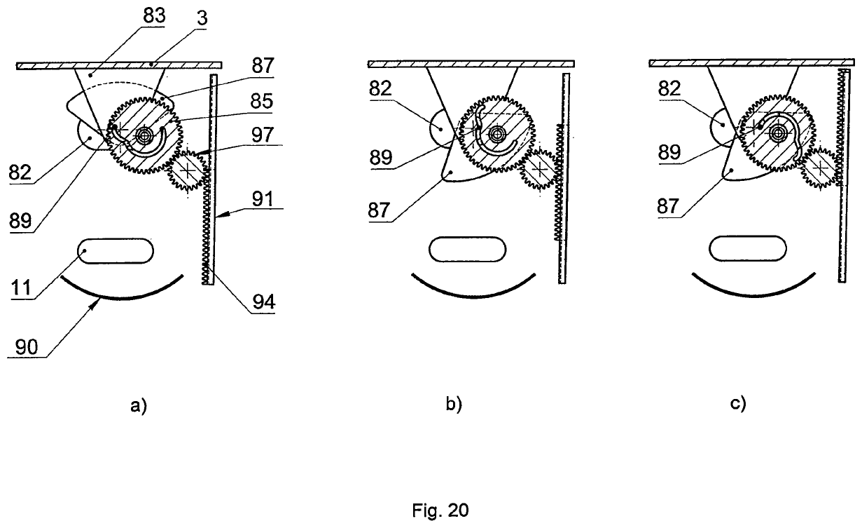

A fixed bracket 83 is disposed above the lamp 82, on the back wall 3 of the housing 1, and an electrical rotation device is disposed above said fixed bracket 83. In this embodiment, said electrical rotation device is manifested as a servo drive 84 of the sterilisation unit, whose hosing is fixedly attached to the back wall 3 of the housing 1 (the mounting brackets are not shown in the drawings). The rotor of the servo drive 84 is connected to a gear 85, which is located on a horizontal plane below said servo drive's housing and configured as a disk mortise cam (FIG. 19, FIG. 20). The aperture of the fixed bracket 83 accommodates a rotatably disposed rotatable sleeve 86, this latter being rigidly connected to a movable bracket 87 of the mirror. The upper end face of the rotatable sleeve 86 is disposed directly against the lower surface of the gear 85. The movable bracket 87 is configured as a flat horizontal plate, with a movable mirror 88 fixedly attached to its lower surface. The movable mirror 88 is configured as a concave, vertically elongated rectangular plate used to reflect and direct lamp's 82 radiation. Inside the rotatable sleeve 86, offset from its centre, there is disposed a vertical cylindrical pin 89, with the lower portion thereof coupled to a movable bracket 87 of the mirror, and with the upper portion thereof disposed in a mortise of the gear 85. A fixed mirror 90, fixedly attached to the outer surface of the frontal wall 2 of the housing 1, is disposed facing the movable mirror 88 (fastening elements are not shown in the drawings). The fixed mirror 90 is also configured as a concave, vertically elongated rectangular plate used to reflect and direct lamp's 82 radiation.

The unit 9 for sterilising the handle levers also comprises a movable curtain 91 used for covering the larger part of the rectangular aperture 5 in the sidewall 4 of the housing 1 to prevent lamp's 82 radiation from leaking outside the housing 1. The movable curtain 91 is configured as a vertically elongated rectangular plate that is located on the rail 92 and rail 93 disposed on the inner surface of the sidewall 4 of the housing 1. That means that the movable curtain 91 is configured to move horizontally along the sidewall 4 of the housing 1 to open and close the larger portion of the aperture 5. Further, the surface of the movable curtain 91, facing the cavity of the housing 1, is fitted with horizontally located upper cogged rack 94 and lower cogged rack 95.

A vertical rotating shaft 96 is rotatably disposed beside the movable curtain 91 on the side wall 4 of the housing 1. The gear 97 is rigidly mounted on the upper part of the rotatable shaft 96, said gear 97 forming a gear transmission with the gear 85, where gear 85 is a master one and gear 97 is a driven one. The upper gear 98 and the lower gear 99 are rigidly mounted on the rotatable shaft 96. The upper gear 98 is configured to form a rack gear transmission with the upper cogged rack 94 of the movable curtain 91. The lower gear 99 is configured to form to form a rack gear transmission with the lower cogged rack 95 of the movable curtain 91.

Thus, the above unit 9 for sterilising the handle levers is configured to open the aperture 5 in the sidewall 4 of the housing 1 by means of drawing the movable curtain 91 and simultaneously cover the lamp 92 with the movable mirror 88. Also, the unit 9 for sterilising the handle levers is configured to close the aperture 5 in the sidewall 4 of the housing 1 by means of drawing the movable curtain 91, and a simultaneously return the movable mirror 88 to its initial operating position.

This configuration of the unit 9 for sterilising the handles is done considering the potential harm of germicidal lamps for human eyesight and skin, and the necessity to protect the consumers from that radiation. Yet, as previously stated, in this case it is not possible to temporarily turn off the germicidal lamp. That is the reason why the protection against said radiation, emitted when the positions of handle levers are changed, is implemented by using said movable curtain 91 and movable mirror 88.

In its operating position, the movable mirror 88 is disposed behind the lamp 82, beside the back wall 3 of the housing 1, as shown in drawing a) FIG. 20. As the gear 85, configured as a disk mortise cam, rotates clockwise, the cylindrical pin 89, sliding in the gear's 85 mortise, starts to move. This brings the movable bracket 87, bearing the movable mirror 88, into anticlockwise rotation. As shown in drawings b) and c) FIG. 20, the pin 89 travels through portion of the gear's 85 mortise and stops, as the movable mirror 88 turns to a position of covering the lamp 82. While the gear 85 rotates further, this cam mechanism performs a no-load run, and hence the movable mirror 88 does not move until the gear 85 makes a full revolution. As the gear 85 starts to rotate, the driven gear 97 together with the rotatable shaft 96 rotate as well. Then, the upper gear 98 and the lower gear 99 of the shaft 96, acting through the upper cogged rack 94 and the lower cogged rack 94, move the movable curtain 91, that slides along rail 92 and rail 93, towards the side of the back wall 3 of the housing 1. Thus, in a full revolution of the gear 85, the aperture 5 in the sidewall 4 of the housing 1 fully opens, as shown in drawings a), b) and c) FIG. 20. When the gear 85 rotates in the opposite direction, the movable mirror 88 returns in reverse order to its initial operating position, and the aperture 5 in the sidewall 4 of the housing 1 is closed by the movable curtain 91.

When the self-sterilising door lever handle assembly is in its normal static state, one of the above-mentioned handle levers is in its operating position, which allows to secure this lever relative to the supporting rotatable sleeve 10, and to rotate the latter when the grasping portion of this handle lever, that protrudes outside the housing 1 through the aperture 5, is pressed. At that time, the second handle lever is in the sterilisation position, i.e inside the housing 1 in the area adjacent to the unit 9 for sterilising the handle levers, and the movable mirror 88 is in its operating position mentioned, while the aperture 5 in the sidewall 4 of the housing 1 is closed with the movable curtain 91. As described above, in this particular embodiment of the present invention, while the handle lever is in an operating position, its thrust pin leans on the support pin 37 of the supporting rotatable sleeve 10.

The electronic control unit 6, mentioned above, is embodied in the form of a printed circuit board (not shown in the drawings) that comprises a microcontroller (not shown in the drawings) and is connected by wires (not shown in the drawings) to the above-mentioned elements: the device for detecting the position of handle levers and electrical rotation devices of the unit 7 for rotating the handle levers, the electric drive for the lock blocking unit 8, and the electrical rotation device of the sterilisation unit 9. In other words, in this particular embodiment, the electronic control unit 6 is configured to have wire connections with the sensing elements of the sensor 27, the servo drive 62, the servo drive 66, the servo drive 70, and the servo drive 84.

The electronic control unit 6 is also configured to detect the position of the handle 11 frontal lever and of the handle 12 back lever using a device for detecting the position of handle levers, and in this particular embodiment—through the sensing elements of the sensor 27.

The electronic control unit 6 is configured to issue rotate commands, any of both directions, to the electrical rotation devices of the unit 7 for rotating the handle levers and the electrical rotation device of the sterilisation unit 9, as well as block and unblock commands to the electric drive of the lock blocking unit 8. That is to say, in this particular embodiment, the electronic control unit 6 is configured to issue rotate commands, any of both directions, to the servo drive 62 of the mechanism for rotating the handle 12 back lever, the servo drive 66 of the mechanism for rotating the handle 11 frontal lever, the servo drive 70 of the lock blocking unit 8, and the servo drive 84 of the unit 9 for sterilising the handle levers.

The principal distinction of the claimed invention is configuring the electronic control unit 6 to issue separate sequential rotate commands to the electrical rotation devices of the unit 7 for rotating the handle levers that enable one handle lever to turn from the operating position to sterilisation position, followed by the turn of the other handle lever from sterilisation position to operating position. Such capability allows minimal eventual negative impact of external forces on mechanisms, which may cause damage to the internal movable parts and software errors in devices. This introduces an anti-vandal protection and eliminates the need to employ robust elements of mechanisms, which fact allows the use of low-capacity and small-sized drives, making the entire device significantly smaller and lighter.

Also, the electronic control unit 6 is configured to issue said rotate commands to the electrical rotation devices of the unit 7 for rotating the handle levers in a programmable time interval and/or after detecting the change in position of the

handle lever that is in the operating position as a result of applying external forces onto the latter, with the supporting rotatable sleeve 10 set in rotation.

Besides, the electronic control unit 6 is configured to issue a rotate command to the electrical rotation device of the sterilisation unit 9 in order to open the aperture 5 in the sidewall 4 of the housing 1, before the above rotate commands are issued to the electrical rotation devices of the unit 7 for rotating the handle levers. Also, the electronic control unit 6 is configured to issue an opposite direction rotate command to the electrical rotation device of the sterilisation unit 9 in order to close the aperture 5 in the sidewall 4 of the housing 1 after completion of previous rotate commands, issued to the electrical rotation devices of the unit 7 for rotating the handle levers.

In addition, the electronic control unit is configured to issue a block command to the electric drive of the lock blocking unit once it is detected that one of the handle levers is in a static position that corresponds to the “blocking” position, and to issue an unblock command once it is detected that one of the above handle levers is in the operating position.

In addition, the electronic control unit 6 is configured to issue a block command to the electric drive of the lock blocking unit 8 once it is detected that one of the handle levers is in a static position that corresponds to the “blocking” position, and to issue an unblock command once it was detected that one of the above handle levers is in the operating position. In this particular embodiment, the electronic control unit 6 is configured to issue a block command to the servo drive 70 of the lock blocking unit 8 once it is detected that the handle 11 frontal lever or the handle 12 back lever is in a static position at an angle to the horizontal line greater than 0° and lesser than 90°, and to issue an unblock command once it is detected that one of said handle levers is in a horizontal position.

The above self-sterilising door lever handle assembly may be installed on hinged doors with any conventional locking device that has a latch, or with a mortise lock with an bevelled latch bolt and a door handle spindle. That is, the self-sterilising door lever handle assembly is to be affixed, as a conventional rotatable handle piece, to a door that has a previously installed locking device with its own door handle spindle.

As discussed above, the present invention offers a configuration of the coupling sleeve 14 of the supporting rotatable sleeve 10 having a through aperture with a

cross-section adapted for coupling to a door handle spindle and for rotating the supporting rotatable sleeve 10 together with said spindle. In a specific embodiment of the present invention, the sleeve 14 has a square aperture adapted for coupling with a conventional spindle of the locking device that has an 8 mm square cross-section. Yet, depending on shape of the cross section of the spindle, said coupling sleeve 14 may be configured with an aperture of another cross section, for example, a square with a side adapted for coupling to a door handle spindle with a 7 mm square cross-section.

The housing 1 of the self-sterilising door lever handle assembly may be affixed to a door by means of any fasteners, particularly threaded ones. Once affixed to a door, the door lever handle assembly is connected via a cable to a relevant power supply unit, connected to the AC mains. Said power supply unit may be mounder, for example, on a door or in the housing 1 of the door lever handle assembly. In a specific embodiment of the present invention, the door lever handle assembly may contain an additional emergency electrical battery in the housing 1.

The above self-sterilising door lever handle assembly operates in a way as follows.

In the initial state of the door lever handle assembly, one of the handle levers mentioned is in the operating position. That is, as discussed above, the grasping portion of said handle lever protrudes outside the housing 1 through the aperture 5, and is disposed horizontally. At the same time, the second handle lever is disposed in the sterilisation position, i.e. inside the housing 1 in an area beside the unit 9 for sterilising the handle levers. The movable mirror 88 is in the operating position, i.e. behind the lamp 82, beside the back wall 3 of the housing 1. The lamp 82 performs sterilisation of this handle lever by irradiation. Meanwhile, the aperture 5 in the sidewall 4 of the housing 1 is covered by the movable curtain 91. The supporting rotatable sleeve 10 of the unit 7 for rotating the handle levers is in the unblocked state, meaning that the latch 74 of the lock blocking unit 8 is out of the mortise 79 of the supporting rotatable sleeve 10.

To open the door by means of activating the lock mechanism, the consumer only has to press the grasping portion of the handle lever that is in the operating position, i.e. turn said lever clockwise. After unlocking the locking device, the handle lever engaged returns, together with the supporting rotatable sleeve 10, into the previous position with help of the return spring 15. I.e., in this case, the door lever handle assembly operates as a regular door handle.

The self-sterilising door handle assembly is configured to block the door locking mechanism to prevent opening the door from the opposite side. The blocking is possible due to the mechanism of the lock blocking unit 8. To perform the blocking, the handle lever, which is in the operating position, should be turned anti-clockwise into the blocking position. In a specific embodiment of the present solution, the blocking position means static position of the handle 11 frontal lever or the handle 12 back lever at an angle to the horizontal line greater than 0° and lesser than 90°.

Meanwhile, as discussed before, the electrical rotation devices of the unit 7 for rotating the handle levers, i.e. the servo drive 62 and servo drive 66, in this particular embodiment, are provided with a magnetic latch for securing the rotor's position when the motors are inactive. That is, when the engines are inactive, the handle levers are in a secured position due to gear transmissions. Thus, in order to rotate a handle lever that is in the operating position one must overcome resistance of the relevant above-mentioned safety device of the unit 7 for rotating the handle levers. The relevant above-mentioned spring safety coupling of the safety device is responsible for securing a handle lever in the blocking position. Securing a handle lever in such a position actually enables the electronic control unit 6 to detect the position of a handle lever as the “blocking position”.

Once a handle lever has been secured in the blocking position, the mechanism of the lock blocking unit 8 actuates, the latch 74 of the lock blocking unit 8 makes a return motion, enters the mortise 79, and thus the position of the supporting rotatable sleeve 10 is secured. The possibility to unlock the door's lock mechanism is thereby eliminated.

In order to unblock the door locking mechanism, one should rotate the handle lever clockwise until it returns to its operating position. The electronic control unit 6 detects that the handle lever has returned to its operating position, the mechanism of the lock blocking unit 8 actuates, the latch 74 of the lock blocking unit 8 makes a return motion and travels out of the mortise 79, and thus the supporting rotatable sleeve 10 is unblocked.

Considering the structure discussed above, the lock blocking unit 8 is adapted to prevent overload and destruction caused by unexpected consumer actions. In particular, if the supporting rotatable sleeve 10 rotates after the blocking mechanism was engaged under the external forces exerted thereon, the blocking

latch 74 will not move into the mortise 79, hitting the external surface of the supporting rotatable sleeve 10 instead. However, once the external force ceases, the supporting rotatable sleeve 10 will return into its normal position driven by the return spring 15. Then, the blocking latch 74 will be automatically conveyed to the mortise 79 of the supporting rotatable sleeve 10 under the influence of the spring 78.

According to the intended use of the door lever handle assembly, it automatically changes positions of handle levers in order to maintain sterility of their grasping portions by way of sequential sterilisation of said levers in the sterilisation unit 9. As previously stated, in a specific embodiment of the present invention, the changes of positions of handle levers, i.e. the turn of one handle lever from its operating position to sterilisation position and the turn of another handle lever from sterilisation position into operating position, may occur in a in a programmable time interval.

Additionally, positions of the handle levers may change once it is detected that the operating position of a handle lever has changed under the influence of external forces actuating the rotation of the supporting rotatable sleeve 10, particularly, after unlocking the door locking mechanism.

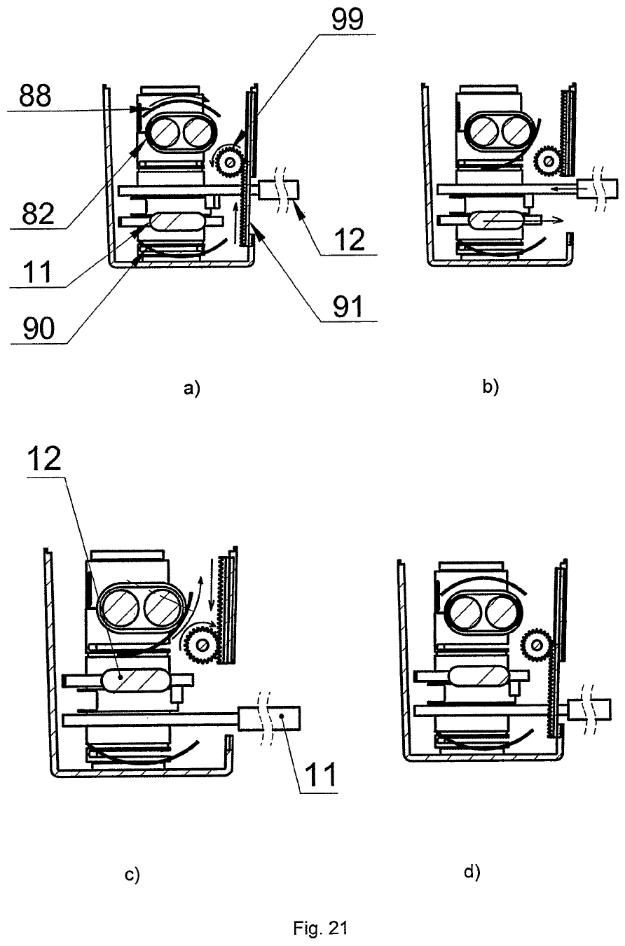

An important feature of the present invention is that the positions of the handle levers change in a sequence. At first, one handle lever turns from operating position to sterilisation position, and only once the sterilisation is completed, the other handle lever turns from sterilisation position to operating position. The sequence of changes of positions of handle levers is shown in drawings a), b), c) and d) FIG. 21.

Said changes of the positions of handle levers occur after the movable curtain 91 has been moved automatically and the aperture 5 in the sidewall 4 in the housing 1 has opened. As the movable curtain 91 moves, the movable mirror 88 turns into a position of covering the lamp 82. Thus, the negative impact of a germicidal lamp's 82 radiation is eliminated as it cannot travel outside the housing 1.

Once the position of handle levers has changed, the movable curtain 91 is moved automatically, covering the aperture 5 in the sidewall 4 in the housing 1. As the movable curtain 91 moves, the movable mirror 88 turns in a reverse order and returns to its operating position.

The self-sterilising door lever handle assembly is configured to actuate the discussed above safety devices of the mechanisms of handle levers, should certain external forces be exerted onto the handle levers while said handle levers are in the secured position, or while said levers are moving to change their position, as described earlier. In a specific embodiment, said safety mechanisms, each of which is configured on the basis of a spring safety coupling, are activated when the external force exceeds the resistance of a relevant spring safety coupling. Said safety devices interrupt the mechanical transmission, protecting the elements of mechanisms from overload and destruction. That is, said safety devices limit the torque that may be transmitted from relevant handle levers.

As can be seen, the above-described structure of the self-sterilising door handle assembly using ultraviolet radiation ensures minimal device dimensions in conjunction with relevant protection of consumers against ultraviolet radiation and protection of mechanisms against damage consequent to application of external forces onto the open movable parts.

What is claimed is:

1. A self-sterilising door lever handle assembly, comprising:
a protective housing with a side aperture,
a mechanism, disposed inside said housing, for advancing and retracting a handle lever through said aperture,
an ultraviolet sterilisation unit disposed inside said housing, and
an electronic control unit disposed inside said housing, wherein
said self-sterilising door lever handle assembly has a unit for rotating the handle levers as the mechanism for advancing and retracting the handle lever through said aperture, and further includes a lock blocking unit disposed in said housing,
said unit for rotating the handle levers is disposed in the lower part of the housing,
said lock blocking unit is disposed on the side of the unit for rotating the handle levers,
said sterilisation unit is disposed above the unit for rotating the handle levers, at the level of the handle levers in their vertical position.
said unit for rotating the handle levers comprises a supporting rotatable sleeve, that serves as the basis of a mechanism for rotating the handle frontal lever and a mechanism for rotating the handle back lever,
the elements of said mechanisms (said elements rotating around the supporting rotatable sleeve) are symmetrically arranged in parallel planes, said planes being perpendicular to the symmetry axis of the supporting rotatable sleeve,
below the supporting rotatable sleeve there are two safety devices, through which the parts of the mechanism for rotating the handle frontal lever and the mechanism for rotating the handle back lever (said parts of the mechanisms directly rotating around the supporting rotatable sleeve) are connected to individual electrical rotation devices through gear transmissions, i.e. every mechanism for rotating the handle lever comprises its own separate safety device and its own separate electrical rotation device,
said safety devices are capable of ensuring forced rotation of the mechanism's elements, should any additional loads thereon occur from the mechanism's elements that directly rotate around the supporting rotatable sleeve,
said supporting rotatable sleeve is rotatably mounted and has a co-axial aperture with a cross section adapted for coupling to a spindle of the locking device and for rotating the supporting rotatable sleeve together with said spindle,
said supporting rotatable sleeve is connected to a return spring configured to return said sleeve into its initial position after rotation,
said parts of the mechanisms for rotating the handle levers that directly rotate around the supporting rotatable sleeve are manifested as
a gear of the handle frontal lever, said gear being rigidly connected to the handle frontal lever, and
a gear of the handle back lever, said gear being rigidly connected to the handle back lever,
said unit for rotating the handle levers has a means of securing the handle levers relative to the supporting rotatable sleeve enabling said sleeve to rotate when the grasping portions of said levers are pressed, said means of securing the handle levers being implemented by ensuring the interaction of special ridges on the surfaces of the supporting rotatable sleeve, handle frontal lever, and handle back lever,
said unit for rotating the handle levers is provided with a device for detecting the position of handle levers,
said lock blocking unit is manifested in the form of a mechanical locking device with a latch that blocks the rotation of the supporting rotatable sleeve, the movement of said latch is enabled by a respective electric drive,
said sterilisation unit contains a germicidal lamp with ultraviolet radiation, an electrical rotation device, a movable mirror, a fixed mirror, and a movable curtain for closing the larger portion of said side aperture in the housing,
said fixed mirror is disposed facing the germicidal lamp and on the opposite side (relative to said lamp) from the area in which the grasping portions of the handle levers are disposed in their vertical position,
said movable mirror in its operating position is disposed on the opposite side from the germicidal lamp (relative to the area in which the grasping portions of the handle levers are disposed in their vertical position),
said movable mirror and said fixed mirror are disposed to be capable of reflecting and directing the radiation of the germicidal lamp onto the area in which the grasping portions of the handle levers are disposed in their vertical position,
said movable mirror is configured to rotate around the germicidal lamp in order to prevent radiation from the germicidal lamp leaking through the open side aperture of the housing,
said movable curtain is configured to move whereby opening and closing a larger portion of the side aperture in the housing to prevent radiation from the germicidal lamp leaking outside the housing,
said movable mirror and said movable curtain are mechanically coupled to the electrical rotation device of the sterilisation unit and are synchronised so that when the movable curtain moves to open the side aperture in the housing, the movable mirror rotates around the germicidal lamp, preventing radiation from the germicidal lamp leaking through the open side aperture of the housing. And vice versa, when the movable curtain moves to close the side aperture in the housing, the movable mirror rotates and returns to its operating position,
said self-sterilising door lever handle assembly is so configured that, while in the static state, one of said handle levers remains in its operating position to enable securing said lever relative to the supporting rotatable sleeve and to rotate the latter when the grasping portion of said handle lever protruding outside the housing through the side aperture is pressed, meanwhile, the second handle lever is disposed in the sterilisation position, i.e. inside the housing in the sterilisation unit area, the movable mirror is disposed in the above-mentioned operating position, and the side aperture in the housing is closed by the movable curtain,
said electronic control unit is embodied in the form of a printed circuit board that comprises a microcontroller, said electronic control unit being connected to the device for detecting the position of handle levers, electrical rotation devices of the unit for rotating the handle levers, electric drive of the lock blocking unit, and electrical rotation device of the sterilisation unit,
said electronic control unit is configured to detect the position of the handle levers using the device for detecting the position of handle levers,
said electronic control unit is configured to issue rotate commands, any of both directions, to the electrical rotation devices of the unit for rotating the handle levers and the electrical rotation device of the sterilisation unit, as well as block and unblock commands to the electric drive of the lock blocking unit,
said electronic control unit is configured to issue separate sequential rotate commands to the electrical rotation devices of the unit for rotating the handle levers that enable one handle lever to turn from the operating position to the sterilisation position, followed by the turn of the other handle lever from the sterilisation position to the operating position,
said electronic control unit is configured to issue said rotate commands to the electrical rotation devices of the unit for rotating the handle levers in a programmable time interval or after detecting the change in position of the handle lever that is in the operating position as a result of applying external forces onto the latter, with the supporting rotatable sleeve set in rotation,
said electronic control unit is configured to issue a rotate command to the electrical rotation device of the sterilisation unit in order to open the side aperture in the housing, before the above rotate commands are issued to the electrical rotation devices of the unit for rotating the handle levers,
said electronic control unit is configured to issue an opposite direction rotate command to the electrical rotation device of the sterilisation unit in order to close the side aperture in the housing after completion of previous rotate commands, issued to the electrical rotation devices of the unit for rotating the handle levers,
said electronic control unit is configured to issue a block command to the electric drive of the lock blocking unit once it is detected that one of the handle levers is in a static position that corresponds to the “blocking” position, and to issue an unblock command once it is detected that one of the above handle levers is in the operating position.

2. The self-sterilising door lever handle assembly according to claim 1, wherein
said means of securing the handle levers relative to the supporting rotatable sleeve is implemented by providing the supporting rotatable sleeve with a support pin on its outer surface, and by providing the handle levers with thrust pins,
said thrust pins are disposed to enable leaning on said support pin and holding the relevant handle levers in a horizontal position, and to enable rotation of the supporting rotatable sleeve when the grasping portions of said levers are pressed with a force exceeding the resistance of the above-mentioned return spring of the supporting rotatable sleeve,
each safety device of the unit for rotating the handle levers is of a spring safety coupling type and is assembled on a shaft configured to rotate so that its rotation axis be parallel to the rotation axis of the supporting rotatable sleeve,
a drive's gear is rigidly mounted on each such shaft, said drive's gear being driven relative to the relevant electrical rotation device of said unit,
a gear of the safety coupling, that is a master gear relative to the respective above-mentioned gear of the handle lever, is rotatably mounted and abuts with one of its end-faces against the thrust collar of the shaft,
a half-coupling is installed directly against the second end face of said master gear, clamped with a spring,
the matching end-faces of the gear of the safety coupling and those of the half-coupling are provided with correspondent matching mortices, with balls disposed between said mortises,
said half-coupling is provided with a feature preventing rotation relative to the shaft's surface,
said half-coupling is configured to move along the shaft if there is a force that exceeds the force of the spring clamping of said half-coupling to said master gear, and to return into its initial state when said force ceases,
said gear of the safety coupling is configured to rotate in conjunction with the shaft, on which it is mounted, and to transmit the torque of the gear of the relevant handle lever if there is no external force that exceeds the force of the spring clamping of said half-coupling,
said device for detecting the position of handle levers of the unit for rotating the handle levers is configured on the basis of a magnetic resistant sensor and is disposed inside the supporting rotatable sleeve,
said device for detecting the position has two sensing elements with one magnet in a non-metal sleeve opposing each of the sensing elements,
each said non-metal sleeve is rigidly connected to one of said handle levers, enabling simultaneous rotation around the symmetry axis of the supporting rotatable sleeve together with a relevant handle lever,
said connection of each non-metal sleeve is carried out via a bracket of the relevant handle lever, drawn through the slot opening in a wall of the supporting rotatable sleeve, each said bracket extends outside the supporting rotatable sleeve through a slot opening and connects to a relevant handle lever,
said slot openings of the supporting rotatable sleeve are configured to enable a 90° rotation of said brackets,
the electrical rotation devices of the unit for rotating the handle levers are embodied as servo drives with a latch for securing the rotor's position when an engine does not operate,
the rotor of each said servo drive is rigidly connected to a gear that forms a gear transmission with the relevant driven gear of said safety device of the unit for rotating the handle levers.

3. The self-sterilising door lever handle assembly according to claim 1, wherein
said electric drive of the lock blocking unit is configured as a servo drive, with a rotor rigidly connected to an adapter sleeve in which a vertical rotating shaft is rotatably disposed, with a latch rigidly fastened to said shaft,
a spring is installed on said shaft, with one end of said spring fastened on the above adapter sleeve and another end fastened on said shaft, in other words, said rotating shaft is adapted for rotation due to its connection to a rotor of said servo drive through a spring,
the lock blocking unit is disposed to enable, while turning into the blocking position, retraction of said latch into a special mortise on the external surface of the above-mentioned supporting rotatable sleeve.

4. The self-sterilising door lever handle assembly according to claim 1, wherein
the sterilisation unit contains a bracket with a socket, in which the above-mentioned germicidal lamp is installed,
above said germicidal lamp, a fixed bracket is disposed, and above the latter there is disposed an electrical rotation device configured as a servo drive, the rotor of said servo drive being connected to the master gear of the drive, said master gear being located in the horizontal plane below the housing of said servo drive and configured in the form of a disk mortise cam,
a rotating sleeve is rotatably disposed in the aperture of the fixed bracket, and rigidly connected to a movable bracket, with the above-mentioned movable mirror fastened on the lower portion of said movable bracket,
inside the rotating sleeve, offset from its centre, there is disposed a vertical cylindrical pin, with the lower portion thereof coupled to said movable bracket, and with the upper portion thereof disposed in a mortise of said drive's gear,
said movable curtain of the sterilisation unit is embodied as a plate installed in the rails disposed on the inner surface of the sidewall of the housing, i.e., the movable curtain is configured to move horizontally along the sidewall of the housing,
the surface of the movable curtain, facing the housing's cavity, is fitted with two horizontal cogged racks,
a vertical rotating shaft is rotatably mounted, beside the movable curtain, on the sidewall of the housing,
on the upper portion of said rotating shaft is a rigidly fixed driven gear that forms a gear transmission with the above-mentioned drive's gear and is a driven one,
two gears forming a racked gear transmission with the cogged racks of the movable curtain are rigidly mounted on said rotating shaft,
said sterilisation unit is so configured that the drive's gear, embodied as a disk mortise cam, when rotating clockwise, moves the above cylindrical pin, which slides in a mortise of said gear,
at the same time, the movable bracket rotates with the movable mirror,
as the movable mirror rotates into the position of closing the germicidal lamp, the cylindrical pin travels a portion of the mortise of the drive's gear and stops,
further rotation of the drive's gear causes a no-load run of said cam mechanism,
that is why the movable mirror does not move until a completion of the full rotation by the drive's gear,
concurrent with the start of rotation of the drive's gear, the above-mentioned driven gear rotates together with the rotating shaft that moves the movable curtain through the above-mentioned cogged racks, so that the side aperture in the housing fully opens in one full rotation of the drive's gear,
when the drive's gear rotates in the opposite direction, the movable mirror returns in a reverse order into its initial operating position, and the side aperture in the housing is closed by the movable curtain.

5. The self-sterilising door lever handle assembly according to claim 1, wherein
said electronic control unit is configured to issue a block command to the electric drive of the lock blocking unit once it is detected that one of the handle levers is in a static position at an angle to the horizontal line greater than 0° and lesser than 90°, and to issue an unblocking command once it is detected that one of the above handle levers is in a horizontal position.
